(19) United States
(12) Patent Application Publication
(10) Pub. No.: US 2005/0245826 A1
(43) Pub. Date: Nov. 3, 2005

(54) Apparatus for imaging human tissue

(76) Inventor: Chetley Livingston Christopher Gervais, Amherstburg (CA)

Correspondence Address: BUTZEL LONG;DOCKETING DEPARTMENT, 100 BLOOMFIELD HILLS PARKWAY, SUITE 200, BLOOMFIELD HILLS, MI, 48304, US

(21) Appl. No.: 11/087,456

(22) Filed: Mar. 23, 2005

Related U.S. Application Data
(60) Provisional application No. 60/555,426, filed on Mar. 23, 2004.
(60) Provisional application No. 60/568,303, filed on May 5, 2004.

(51) Int. Cl. A61B 8/00

(52) U.S. Cl. 600/443; 128/916

(57) ABSTRACT

An apparatus for imaging human tissue includes a piezoelectric crystal probe mounted on a frame for generating imaging data. The probe can be a linear array movable relative to the frame or a fixed matrix.

### CROSS-REFERENCE TO RELATED APPLICATION

This application claims the benefit of U.S. provisional patent application serial No. 60/555,426 filed on Mar. 23, 2004 and the benefit of U.S. provisional patent application serial No. 60/568,303 filed May 5, 2004.

### BACKGROUND OF THE INVENTION

The present invention relates generally to an apparatus for imaging and, in particular, to an apparatus for imaging the human body.

Except for skin cancer, breast cancer is the most commonly diagnosed cancer among American women. It is second to lung cancer as the leading cause of cancer-related death among women. In 2003, an estimated 211,300 new cases of invasive breast cancer were diagnosed among women. In 2003, an estimated 39,800 women died of this disease. If detected early, the 5-year survival rate for localized breast cancer is 97%.

Breast cancer diagnosis is presently based on clinical examination, mammography and two dimensional (2-D) breast ultrasound examination. Since its introduction as a research tool in the 1950's, ultrasound has steadily progressed towards its current status as an indispensable modality in the diagnosis of many diseases. Recently, higher frequency ultrasound transducers have dramatically improved the visualization of soft tissues. This has increased its value during musculoskeletal, small parts and breast ultrasound investigations.

The utility of breast ultrasound however remains limited for a number of reasons including because conventional ultrasound images are 2-D, a limited number of images can be reasonably submitted during each breast ultrasound examination. Therefore, the radiologist must attempt to mentally transform selected 2-D images obtained from a variety of perspectives into a “registered” three dimensional (3-D) data set within his mind to make a diagnosis of breast cancer or benign breast disease. In addition, due to non-planar nature of 2-D ultrasound image acquisition, accurate localization or reproduction of a specific image at a later time is not possible. This makes quantitative or follow-up of disease progression or assessment of tumor growth or shrinkage after therapy inaccurate or impossible. Also, normal patient anatomy restricts the available ultrasound probe angles and may frequently make the optimal image plane for disease assessment unavailable. Furthermore, therapeutic planning frequently requires serial volumetric measurements, although these can be obtained via 2-D ultrasound the values are frequently variable and inaccurate. As a result, it is difficult to accurately correlate standard mammographic images with breast ultrasound images except in the most general sense (for example, a mass is “within the upper outer quadrant of the left breast”).

Presently, as mammography is performed with compression in the MOL and CC projections with the patient sitting or standing upright, but breast ultrasound is performed with the patient supine and without compression and with 2-D non-planar images taken from multiple perspectives and/or angles and there really is no accurate way to correlate mammography and breast ultrasound images except in the most general way.

For example, a radiologist may see a suspicious “mass” on two views, then localizes it to “approximately X centimeters from the nipple at 10 o'clock”. After this, a breast ultrasound is then performed and all the technician can actually say (based on the different patient positions, angles of imaging and lack of compression) is that they do or do not see a mass within the same quadrant of the breast. This, in the context of breast cancer investigation and decisions regarding biopsy or cancer treatment is clearly sub-optimal.

Conventional ultrasonography is inherently tomographic, like Computed Axial Tomography (CT) Scans, or Magnetic Resonance Imaging (MRI). Theoretically, it is possible to reconstruct a registered 3-D tissue block or voxel array, discussed in more detail below, which can then be used for three-dimensional or multiplanar review. However, unlike CT or MRI during which images are collected at precisely defined intervals at a slow rate of acquisition producing a “stack” of parallel slices, ultrasound images are produced at a high frequency (15-60 frames per second) with the relative orientation of each image in the sequence being arbitrary and under the direct control of the ultrasonographer. In addition, CT and MRI are presently not considered to be either economical or useful in the investigation of breast cancer/breast cancer screening. Image reconstruction or “re-registration” during ultrasound examinations is inherently difficult and over the past two decades there have been many attempts to solve re-registration problems. Reconstruction of 3-D nonparallel images requires accurate integration of specific transducer position information with the acquired two-dimensional ultrasound images. Only in the past few years has the development of high-speed computer processors and enhanced computer memory (both RAM and hard drive space) made three-dimensional ultrasound a possibility. The obvious advantages of 3-D ultrasound over conventional 2-D ultrasound have resulted in a great deal of research in this area recently.

Commercial production of high-end ultrasound equipment with 3-D capability is the most recent result of this process. Unfortunately even the latest commercially available three-dimensional ultrasound technology has limitations. It remains very expensive and continues to visualize only small fields of view. These factors continue to prevent widespread use of this technology.

There have been many attempts to overcome these limitations by using inexpensive and/or readily available conventional 2-D ultrasound equipment to produce 3-D images. Each of these systems requires image re-registration which can only be accomplished by tagging or associating each image with specific information concerning the transducer orientation and/or position in 3-D space. There are two basic mechanisms of obtaining this information, mechanical 3-D scanning devices and sensed free-hand scanning systems. Mechanical 3-D scanning devices offer the potential of both speed and accuracy; however, they are generally bulky and difficult to use except in very specific applications. Sensed free-hand scanning systems are preferred because they allow more natural transducer motion and flexibility. There have been four basic free-hand position sensing techniques developed in the prior art which include: A) articulated arms; B) acoustic tracking; C) magnetic field tracking; and D) image-based information.

The primary difficulty during construction of an accurate 3-D data block is the reregistration of the ultrasound images being obtained. This is easy with CT and MRI where the patient rides on a level track through a scanner at a constant rate so that each slice is already registered with both adjacent slices and multiplanar reconstruction or creation of a 3-D voxel array is not difficult. Also, x-rays and signals produced during MRI can travel through the air. Ultrasound, however, does not travel well through air and a water or gel-to-skin interface is required to image tissue. In standard tissue ultrasound examinations, a 2-D probe is placed against the skin after applying an imaging gel interface to ensure passage of the beam into the underlying tissue and reception of the reflected echoes by the piezoelectric crystal within the ultrasound probe surface. Presently the limitations of 3-D ultrasound probes, high cost of technology, small field of view and limited angles of visualization make this technology of limited use in daily medical practice. It is also not possible to accurately correlate the obtained 3-D images with other modalities such as plain x-ray, CT or MRI.

As previously indicated, current expensive phased array 3-D ultrasound technology provides a very limited field of view, examining only a small section of the breast anatomy at any one time. As there is no way to define where each 3-D image (tissue wedge) is obtained in relation to any other 3-D probe image, this technology has the same basic limitations as 2-D ultrasound. It cannot be precisely correlated with mammography of the same breast. Also there is simply no way to accurately relate two abnormalities within different 3-D tissue wedges. 2-D ultrasound representative images from various perspectives may or may not overlap and actually demonstrate only a very small amount of the actual breast tissue based on the volumes of the thin data slices. The examination is also very operator or technician dependent. It is the technician who decides which two to three dozen thin 2-D slices to save and submit for radiologist review. It is also easy for significant pathology to be missed based on operator skill. In the end, the radiologist only ever sees a very small amount of breast tissue. This is clearly a sub-optimal system of breast tissue review. It would be similar to reviewing a mammogram, a CT image or a MRI image with hundreds of (non-displayed) big holes in it.

It is desirable, therefore, to provide a device with the capability of 3-D multiplanar reconstruction that allows precise ultrasound correlation with a specific mammographic finding.

### SUMMARY OF THE INVENTION

The imaging apparatus in accordance with the present invention provides a novel solution to the problems noted above. The imaging apparatus demonstrates how inexpensive and widely available 2-D medical ultrasound equipment can be used to produce “full field” high resolution multi-planar reconstructed ultrasound slices from a true 3-D data block. These images can be reviewed in any plane, singly or reconstructed then reviewed in sequence (like a CT or MRI scan).

Most recently a Canadian company, the NDI Corporation based in Waterloo Ontario, has developed a highly accurate (0.1 mm at a 2 meter sensor distance) infrared-based device, the “Polaris”. This device has both medical and industrial applications. After calibration, the dual Polaris infrared sensors or eyes are focused on a reflective target pattern, known as a passive tool, that is attached to the ultrasound probe, and these eyes detect tiny alterations in the relative position of the passive tool reflective targets with respect to a fixed (in 3-D space) tool target pattern (the passive tool). This spatial information is then attached to the 2-D image file.

Software, such as the STRADX software program, discussed in more detail below, then assembles the multi-planar raw ultrasound slice data obtained from conventional scanning and, without the creation of a calculated 3-D voxel array, allows visualization of the intersecting points along any scan plane selected by the investigator. This represents true multi-planar reconstruction without the sophisticated mathematical interpolation or voxel calculation required to create a 3-D voxel array as is done with CT or MRI images.

The apparatus in accordance with the present invention creates a voxel array from the 3-D STRADX data block which can be reviewed using a standard medical 3-D imaging workstation (this may require conversion of this data into a DICOM3 medical imaging format). The term voxel is short for volume pixel, the smallest distinguishable box-shaped part of a three-dimensional image. “Voxelization” is the process of adding depth to an image using a set of cross-sectional images known as a volumetric dataset. These cross-sectional images (or slices) are made up of pixels. The space between any two pixels in one slice is referred to as interpixel distance, which represents a real-world distance. The distance between any two slices is referred to as interslice distance, which represents a real-world depth. The dataset is processed when slices are stacked in a computer memory based on interpixel and interslice distances to accurately reflect the real-world sampled volume. Next, additional slices are created and inserted between the dataset's actual slices so that the entire volume is represented as one solid block of data. Now that the dataset exists as a solid block of data, the pixels in each slice have taken on 30 volume and are now voxels. For a true 3-D image, voxels must undergo opacity transformation. Opacity transformation gives voxels different opacity values. This is important when it is crucial to expose interior details of an image that would otherwise be hidden by darker more opaque outside-layer voxels. Voxel images are primarily used in the field of medicine and are applied to X-Rays, CT (Computed Axial Tomography) Scans, and MRIs (Magnetic Resonance Imaging) so that medical professionals can obtain accurate 3-D models of the human body. Voxel imaging is also being used to create computer games, so 3-D acceleration is not necessary.

The imaging apparatus in accordance with the present invention retains the technical advantages of this new breast imaging system but makes practical advances based on manufacturing sophistication and without the use of 3-D position sensing equipment. The essential aspect of the imaging apparatus is a generation of a 3-D ultrasound image based use of a fixed (large field) array of ultrasound transducers, which can provide both rapid and real time 3-D as well as be correlated accurately with digital mammographic images. It also allows exact 3-D positional correlation of all objects within the imaged 3-D data block once created.

In addition, as the imaging apparatus is fully electronic, the creation of this 3-D data block is virtually “instant” limited only by the speed which the selected elements in the large field array can be sequentially fired and the “echo time” off the tissues, which means that (assuming adequate computer processing power) real time 3-D (10-15 feet per second) would be possible using CRT displays, advantageously allowing unparalleled soft tissue imaging of moving and/or vascular structures and even during surgery.

The imaging apparatus also provides for selective visualization of different tissues based on echogenic properties such as brightness of echoes or movement of fluids on doppler examination. This can be done using “auto segmentation”, which is a process whereby only echoes of a specific strength or with specific characteristics will be displayed and (unlike CT or MRI) the imaging apparatus has the potential to do this in real time.

The imaging apparatus in accordance with the present invention advantageously includes a large field array of pre-registered (in 3-D) sequentially fired ultrasound transducers that fixed in their relative positions to each other and are used to create a 3-D data block. This array, in one embodiment, is attached to and/or correlated with the production of a digital mammographic image, and may be used to produce “real time” large field 3-D imaging in conjunction with “tissue segmentation” based on echogenicity (which would allow visualization of an object such as a surgical instrument or endoscope passing through the 3-D tissue block).

The key aspect of the imaging apparatus, of course, is having the piezoelectric crystals fixed in position to each other and sequentially electronically fired to produce the 3-D image. The non-mechanical aspect allows for far more reliable and far faster data collection limited only by the response time for the echos to reach the target tissues and return to the Imaging apparatus or matrix probe element.

In an alternative embodiment, the fixed matrix of ultrasound transducer elements might be curved (either convex or concave) to improve the visualization of certain tissues or materials in 3-D.

The imaging apparatus in accordance with the present invention provides a novel system of “full-field” 3-D breast ultrasound based on existing 2-D ultrasound equipment and advanced NDI 3-D spatial position sensing technology. The imaging apparatus produces multi-planar reconstructed breast tissue images which can be directly correlated with standard mammography images. The imaging apparatus can be installed in conjunction with existing 2-D ultrasound and mammography equipment without expensive modification.

### DESCRIPTION OF THE DRAWINGS

The above, as well as other advantages of the present invention, will become readily apparent to those skilled in the art from the following detailed description of a preferred embodiment when considered in the light of the accompanying drawings in which:

FIG. 1A is a top plan view and FIG. 1B is a cross-sectional side elevation view of an apparatus for imaging human tissue in accordance with the present invention; and

FIG. 2 is a schematic view of an alternate embodiment imaging apparatus according to the present invention.

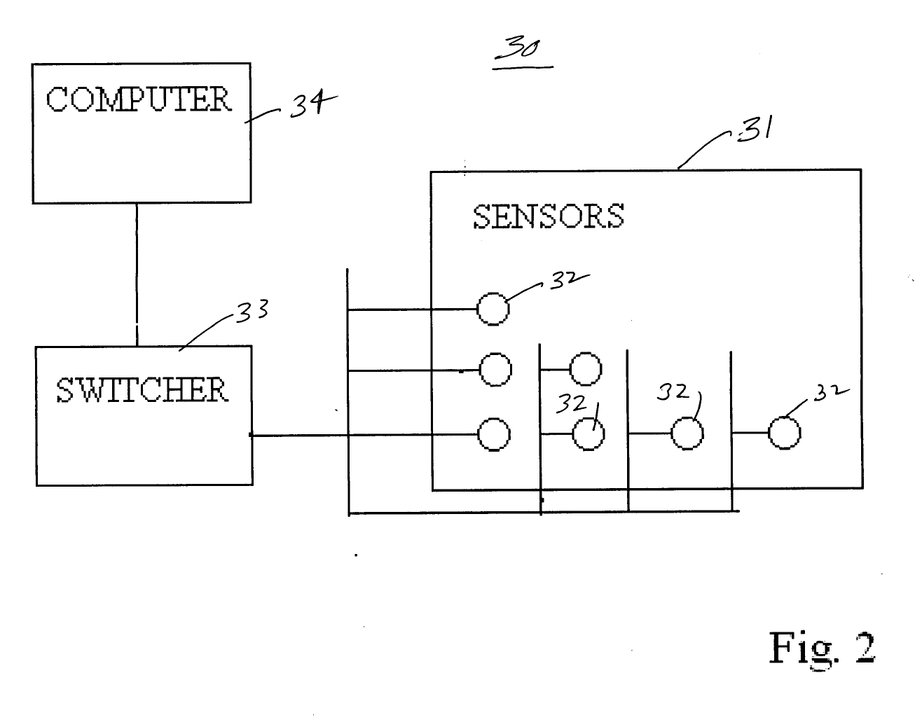

### DESCRIPTION OF THE PREFERRED EMBODIMENT

The U.S. provisional patent application serial No. 60/555,426 filed on Mar. 23, 2004 and the U.S. provisional patent application serial No. 60/568,303 filed May 5, 2004 are incorporated herein by reference.

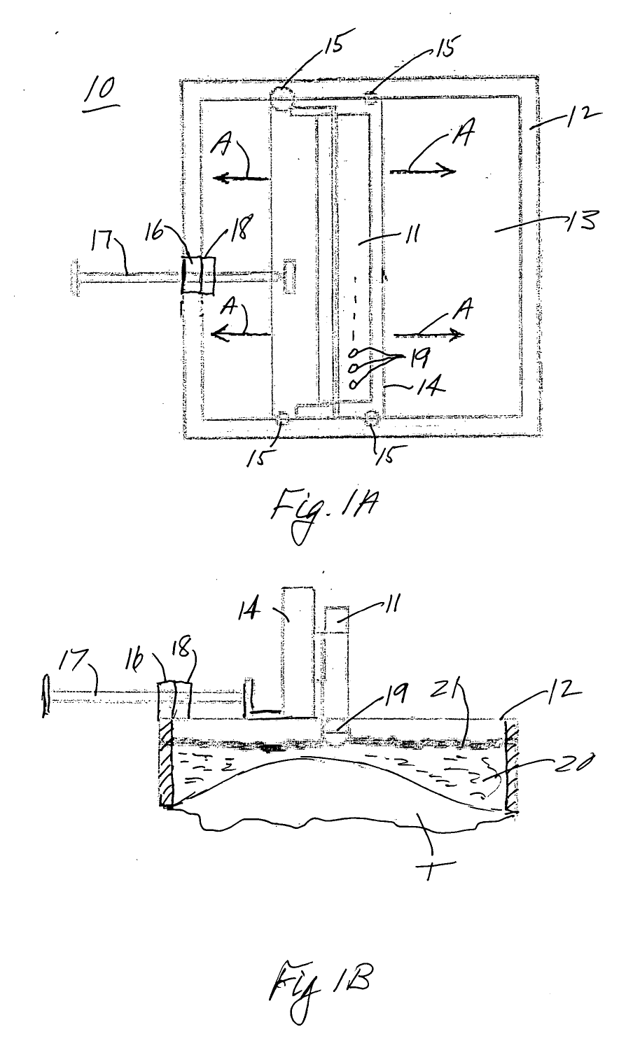

Referring now to FIGS. 1A and 1B, an apparatus for imaging human tissue in accordance with the present invention is indicated generally at 10. A probe 11 is mounted on a frame 12 for movement in the direction of arrows A over an open center 13 of the frame. The probe 11 can be removably attached to a support 14 that rides on balls or wheels 15, or any suitable sliding mechanism. A motor 16 mounted on the frame 12 drives a threaded rod 17 that is connected to the support 14 to produce the desired movement. An encoder 18 is driven by the rod 17 to generate a signal representing the position of the probe 11. The probe 11 can be a linear array of piezoelectric crystal elements 19 that generate signals representing a slice of tissue below the probe. As shown in FIG. 1B, a quantity of ultrasound gel 20 and a gel standoff pad 21 are placed over tissue T, such as a human breast, to be scanned.

The imaging apparatus 10 works like a miniature CT or MRI scanner. This device “manually” creates a large field ultrasound crystal array by recording and “gluing together” slice by slice a “pre-registered” data set (where all the slice widths/heights and linear positions along an axial track are predetermined as parallel and of a uniform thickness) in a manner similar to a mini-CT scanner. The individual slice images would also be created as full DICOM3 medical images by using an advanced ultrasound scanner so they can be stitched together by existing very sophisticated 3-D medical review software for workstation review. This apparatus 10 produces registered data sets without the use of external 3-D position sensors or complex math with data extrapolation (making up points in 3-D space).

An alternate embodiment imaging apparatus 30 includes a “full field” (for example, ten inch by twelve inch) piezoelectric crystal array or matrix probe 31 including a plurality of piezoelectric crystal elements 32. All of the probe crystal elements 32 are “pre-registered” (rows and columns) and are fired in a pre-determined order during use of the matrix probe 31 in order to produce three dimensional (3-D) or four dimensional (4-D, which is 3-D including time) data sets at more than fifteen feet per second (fps). Producing 3-D or 4-D data sets at more than fifteen fps allows for real time 4-D data set review including robotic endosopic surgery in space controlled by surgeons on the ground as well as virtual angiography and more accurate tumor size assessment etc.

Each of the crystals 32 is connected to a switcher 33 that controls the “firing” under the control of a computer 34 that receives the signals generated by the matrix. The computer 34 stores the data from the matrix 31 to generate the image of the tissue that was scanned.

Hardware and software for processing the scan data are discussed in detail in the above-identified provisional patent applications. For example, the STRADX 7.0 3-D ultrasound software is a research tool designed for investigation of free-hand 3-D ultrasound applications. It has primarily been used in obstetrical and organ volumetric applications. As described on the Cambridge Machine Intelligence Group website, the STRADX software is a tool for the acquisition and visualization of 3-D ultrasound using a conventional 2-D ultrasound machine. To record qualitative 3-D ultrasound data with Stradx the basic requirements are a suitable computer with video acquisition facilities, such as a Linux PC with a supported video acquisition card (or a Gage digital input board for RF acquisition), or a SGI O2, Octane or Indy workstation with the appropriate video option, at least 64 and preferably more than 128 MB of memory, a conventional 2-D ultrasound machine with either a video output (PAL, NTSC or custom digital), or access to the analog RF signal.

In the imaging apparatus or matrix probe system enhanced re-registration accuracy is obtained by: 1) providing a stable scan reference plane for probe motion across the patient's skin (The use of a fluid filled “stand-off” pad also greatly reduces deformity of the skin surface during scanning.); and 2) the imaging apparatus or matrix probe system also successfully eliminates the second cause of inaccurate data re-registration—patient motion during the scan.

Breast tissue stabilization in the imaging apparatus or matrix probe system uses a modified Lorad Medical Systems mammography machine paddle. This also produces another technical advantage to this new method of performing a breast ultrasound examination—the breast tissue is being stabilized in exactly the same position as during a standard mammography examination. This means that after creation of a 3-D data block, the imaging apparatus or matrix probe system will allow for multi-planar re-slicing and review of the image data from exactly the same perspective as provided during standard mammography.

This, therefore, represents the first time that a breast ultrasound study can be accurately correlated with a mammography examination of the same breast. The imaging apparatus of multi-modality (mammography/breast ultrasound) image correlation will provide a substantial improvement in the process of breast cancer screening/diagnosis.

The imaging apparatus in accordance with the present invention offers a full-field breast ultrasound examination. Specifically, not only is the entire breast imaged at one same time but also that all breast tissue sections are included within the breast 3-D tissue block obtained. The radiologist is then able to directly review all of the breast tissue in any chosen plane without pre-selection by the technologist. Clearly both the opportunity to perform multi-planar reconstruction (including in a plane which can be directly correlated with standard mammography) and the ability to review all of the breast tissue data represents a substantial improvement over presently available technology.

Equally important during post diagnostic treatment planning and evaluation of breast cancer therapy is the use of ultrasound to perform tumor measurements including volumetric measurements. The response of cancers to radiation or chemotherapy is generally evaluated by measuring tumor size sequentially.

Cancers are not spherical and 2-D ultrasonic measurement is both inaccurate and unreliable when compared with volumetric measurements obtained using 3-D imaging (CT or MRI). This is because 2-D ultrasound cannot be reliably performed from the same position or with the same mass diameters. The imaging apparatus or matrix probe imaging system offers the opportunity to obtain accurate and reliable serial volumetric measurements of both tumors and organs because the variation in observer perspective intrinsic to 2-D ultrasound and manual measurement errors are essentially eliminated by creating a 3-D data set which includes the entire tumor mass. The STRADX software allows for accurate 3-D volumetric calculation based on selecting tumor tissue planes manually on several 2-D slices.

The re-registration problems commonly relating to probe motion in various planes on the patient's skin, are virtually eliminated by the use of a stable plane of reference (the imaging apparatus or matrix probe scanning frame) which both compresses and stabilizes the underlying breast tissue.

Beyond the substantial diagnostic advantages that this imaging apparatus or matrix probe system may provide to the interpreting Radiologist, there are several important economic and public health implications of the use of this new technology. For example, when breast ultrasound is performed by a qualified technician it generally requires a ½ hour time slot and either dual booking or registration of the patient into the Hospital/clinic database. This is not only expensive for the facility to perform but also occupies a substantial amount of skilled technician time during which that technician could be performing other needed ultrasound examinations. The breast ultrasound examination utilizing the imaging apparatus ## can be performed by untrained non-medical staff in less than 5 minutes. Not only does this system offer the opportunity to obtain a dramatically increased amount of diagnostic information but it could increase patient throughput by reducing examination times by over 80%. This would provide both a financial benefit to hospitals and/or clinics. The logical design progression of the imaging apparatus or matrix probe system is to transform the current free-hand 3-D position sensor linked prototype into a true electronic imaging apparatus or matrix probe.

Constructing a “full-field” sequentially firing matrix of piezoelectric crystal elements would take the next step into true real time 3-D (4-D) breast ultrasound. A commercial version of the imaging apparatus or matrix probe system preferably uses electronic sequential scanning over the entire surface of the compression plate. The speed at which this switching could occur would result in “real time” 3-D imaging allowing precise evaluation of both arterial and venous blood flow throughout the breast including any detected tumors. It would retain all of the advantages of the imaging apparatus 10 but eliminate the need for the position sensor 18 (as all probe element positions and orientations are pre-defined and stable for any scan just like CT or MRI).

The computer 34 controls the switcher 33 to acquire data from each of the sensors in a predetermined sequence and at a predetermined cycle rate. Since the sensors are fixed with reference to the tissue being imaged, the acquired data can be used to present a series of images similar to movie or television frames that can provide a 3-D view of, for example, blood flow.

This embodiment of the imaging apparatus also allows it to be directly integrated into a “dual purpose” fully digital mammography/breast ultrasound machine which at the time of mammography with the push of a button would automatically perform a 3-D breast ultrasound without additional technician time or expense. The imaging file at that time would include both the 2-D digital mammography image and the 3-D/4-D ultrasound data block, which could be reviewed on the same workstation and even allow for Radiologist superimposition of image information from both (mammography and ultrasound) modalities.

The apparatus and method according to the present invention involves the creation of a 3-D soft tissue data set using the combination of free hand 2-D ultrasound equipment, a spatial position indicator, a computer algorithm to identify the relative positions of the scan image pixels and, in the original embodiment, tissue stabilization using a frame with probe guidance slots and a standard mammography paddle/machine to reduce the inherent variability of the 2-D scan plans and stabilize the soft tissues.

In the alternate embodiment of the present invention, in order to eliminate the need for the use of an expensive 3-D spatial position sensor (like the NDI Polaris) and avoid the time consuming and very precise calibration procedures required to use this sensor, including creation of passive tools to act as sensor targets and calibration aids (the Cambridge phantom), and scanning with these sensors attached to the ultrasound probe, we are proposing to create a rigid sequentially firing full field ultrasound transducer “matrix (super) probe, based upon a standard mammography paddle to allow for production of pre-registered (in contrast to re-registered) ultrasound 3-D data blocks, based upon the design of the electronic imaging apparatus or matrix probe—for which all of the current free hand spatial variables (except depth of focus, which is more a function of the ultrasound machine driving the imaging apparatus 10) are pre-determined.

Like CT and MRI, if a full field image of the breast tissue could be produced with out any variation in the angle, tilt, or width of the ultrasound beam, each time the scan sequence was performed (if more than 15 per second, it equals a real time 4-D ultrasound) then each 2-D slice would already be registered. So a reconstruction in 3-D or any chosen re-slice plane would be as easy as CT or MRI and review of these images on a standard 3-D review workstation and integration into a standard medical imaging database (PACS system) is facilitated.

Of course the imaging apparatus in accordance with the present invention could be used elsewhere by just being removed from the mammography machine in order to produce larger 4-D images of other applicable soft tissue areas such as the abdomen, the thigh, the calf, etc., as the technology would still allow for high quality imaging in any area with good probe to skin contact with or without a standoff pad, which allows better superficial soft tissue visualization and wider skin contact due to the way a stand off pad would allow ultrasound waves to reach the skin surface where compression directly might not be possible, such as the edge of the breast not being directly compressed by the paddle.

The electronic imaging apparatus or matrix probe in accordance with the present invention is not just a “big probe”, but is rather a large number of parallel piezoelectric transducer elements that have been arranged in order to produce a 3-D or 4-D data block. This could either be by using a series of sequentially firing linear and/or convex transducer elements beside each other or a matrix of phased array probe elements, which produce a small field of view 3-D by using “beam steering” whereby the ultrasound beam is electronically steered rapidly back and forth—this is the basis of the 3-D on most very high end new ultrasound machines)

Preferably, multiple (2-50) such phased array probe elements each producing a small 3-D data block which is then assembled by software into a larger data block. The imaging apparatus or matrix probe 3-D medical ultrasound system uses 2-D equipment to produce a 3-D data block for review and/or reconstruction like a CT or MRI scan. This engineering innovation allows correlation of multi-planar 3-D ultrasound images and standard mammography images improving diagnosis/treatment of breast cancer. This is the same concept as using a construction of linear probe elements to produce that 3-D data block by sequentially firing and reading the linear probe elements.

The present invention relates to the concept of the assembly of multiple linear/convex or phased array transducer elements to produce a pre-registered 3-D image of the soft tissues. The 3-D images are pre-registered because the 3-D spatial relationship between the 2-D images subsequently produced is pre-determined and static allowing for a greatly improved and facilitated 3-D reconstruction of the tissues being imaged. In fact, the resolution of the imaging apparatus is no longer limited by the weakest link in the chain of probe frequency, computer processing power, 3-D spatial calibration, tissue compression adjustment and the vagaries of voxel interpolation (with data creation by mathematical algorithms during “calculation” of the 3-D data block).

With this imaging apparatus, the resolution is only limited by the wavelength of the ultrasound beam. The higher the frequency and shorter the wavelength, the higher the resolution of the ultrasound probe, but the less the wavelength can penetrate soft tissues.

In the first embodiment 10, a single (full width) probe element can be used to sweep over the entire breast (like a broom- either flat or curved to match the radius of the scan head as it moves over the breast around a point in space).

In accordance with the provisions of the patent statutes, the present invention has been described in what is considered to represent its preferred embodiment. However, it should be noted that the invention can be practiced otherwise than as specifically illustrated and described without departing from its spirit or scope.

1. An apparatus for imaging tissue comprising:
an ultrasound 2-D probe;
a scanning frame upon which said probe is mounted for positioning said probe relative to an area of human tissue;
a stand-off pad adapted to be positioned between said frame and the area of human tissue; and
means for generating a 3-D image from signals generated by said probe.

2. The apparatus according to claim 1 wherein said probe is a matrix of a plurality of piezoelectric crystals.

3. The apparatus according to claim 2 wherein said piezoelectric crystals are arranged in rows and columns.

4. The apparatus according to claim 2 including a switcher connected to said piezoelectric crystals for controlling acquisition of data in a predetermined sequence.

5. The apparatus according to claim 4 including a computer connected to said switcher for storing said data.

6. The apparatus according to claim 1 wherein said probe is a linear array of a plurality of piezoelectric crystals.

7. The apparatus according to claim 6 including means for moving said probe relative to said frame in a direction transverse to an axis of said linear array.

8. The apparatus according to claim 7 wherein said apparatus for moving is a motor driving a threaded rod connected to said probe.

9. The apparatus according to claim 6 including means for generating a signal representing a position of said probe relative to said frame.

10. An apparatus for imaging tissue comprising:
an ultrasound 2-D probe having a plurality of piezoelectric crystals arranged in a matrix;
a scanning frame upon which said probe is mounted for positioning said probe relative to an area of human tissue;
a stand-off pad adapted to be positioned between said frame and the area of human tissue; and
means for generating a 3-D image from signals generated by said probe.
